(19) United States
(12) Patent Application Publication
(10) Pub. No.: US 2011/0082495 A1
(43) Pub. Date: Apr. 7, 2011

(54) Apparatus And Methods For Excluding The Left Atrial Appendage

(76) Inventor: Carlos E. Ruiz, New York, NY (US)

(21) Appl. No.: 12/573,025

(22) Filed: Oct. 2, 2009

(51) Int. Cl. A61B 17/08

(52) U.S. Cl. 606/213

(57) ABSTRACT

Apparatus and methods are provided for excluding and reducing the volume of the left atrial appendage (“LAA”) by deploying a first tissue capture element in contact with the pericardium and a second tissue capture element in engagement with the endocardial surface adjacent to the ostium of the LAA, such that the LAA tissue is retained in a collapsed, reduced volume state therebetween. Methods of using the apparatus of the present invention to reduce or occlude the LAA also are provided.

### I. FIELD OF THE INVENTION

This application generally relates to apparatus and methods for excluding the left atrial appendage in humans.

### II. BACKGROUND OF THE INVENTION

Embolic stroke is the one of the nation's leading mortality factors for adults, and is a major cause of disability. A common cause of embolic stroke is the release of thrombus formed in the left atrial appendage (“LAA”) resulting from atrial fibrillation. The LAA is a small windsock-like cavity that extends from the lateral wall of the left atrium generally between the mitral valve and the root of the left pulmonary vein. The LAA normally contracts with the left atrium during systole, thus preventing blood within the LAA from becoming stagnant. During atrial fibrillation, however, the LAA fails to vigorously contract due to the lack of synchronicity of the electrical signals in the left atrium. As a result, thrombus may form in the stagnant blood that pools within the LAA, which may subsequently be ejected into systemic circulation after a normal sinus rhythm is reinstituted.

In a report entitled “Appendage Obliteration to Reduce Stroke in Cardiac Surgical Patients With Atrial Fibrillation,” Ann Thorac. Surg., 1996. 61(2):755-9, Blackshear and Odell found that of 1288 study patients with non-rheumatic atrial fibrillation, 17% had thrombus detected in the left atrium of the heart, and of those patients, in 91% the thrombus was located within the left atrial appendage. That study and others have shown that eliminating or containment of thrombus developed within the LAA of patients with atrial fibrillation may significantly reduce the incidence of stroke in such patients.

As reported in an article in the New England Journal of Medicine, “Left Atrial Appendage Occlusion—Closure or Just the Beginning?,” N. Engl. J. Med 360:25, 2601-2603 (Jun. 18, 2009), the strong association between thrombus formation in the LAA prompted the Food and Drug Administration in late 2008 to grant expedited-review status for clinical testing of the Watchman technology, described in U.S. Pat. No. 6,730,108. The devices described in that patent generally consist of a frame and cover arrangement that blocks the entryway to the LAA. As of the date of that article, no percutaneously deliverable device had been approved for this purpose. As reported in that article, experience implanting the Watchman device was observed to carry substantial upfront procedural risk. After 449 attempted implantations, the device was successfully placed in 408 patients (90.9%). Overall, 12.3% of patients had serious procedural complications, including pericardial effusion requiring drainage or surgery in approximately 5% and acute ischemic stroke due to air or thromboemboli in 1.1%. This experience shows that alternative apparatus and methods for excluding the LAA warrant investigation.

Aside from the Watchman device, other apparatus and methods are described in the prior art for excluding the LAA. For example, U.S. Pat. No. 7,192,439 to Khairkhahan et al, describes an implantable occlusion device that may be deployed to occlude the ostium of the LAA cavity.

U.S. Pat. No. 7,115,110 to Frazier et al. describes apparatus that may be percutaneously inserted into a body cavity and which deploys a series of barbs at the ostium of the cavity. The barbs are subsequently drawn together like a purse string to pull the tissue together, thereby closing off the ostium.

U.S. Pat. No. 7,527,634 to Zenati et al. describes apparatus and methods for closing off the LAA using a pericardial approach, in which a lasso is placed around the base of the LAA and drawn together to close off the entryway to the LAA. U.S. Pat. No. 7,344,543 to Sra similarly describes a device for use with a minimally invasive pericardial approach, in which a detachable coil is applied to the base of the LAA, thereby isolating the cavity.

There are expected to be several drawbacks common to the above-described devices and methods. For example, most of the previously-known percutaneous devices are designed for an ideal LAA anatomical structure, including a well-defined, symmetric, and typically circular ostium and expected depth and orientation of the LAA cavity. The Watchman device, for example, assumes that the ostium to the LAA will be symmetric, and that the orientation of the LAA cavity is substantially perpendicular to the plane of the left atrium. Due to patient-to-patient variability of the LAA anatomy, however, the occlusion surface of that device may not cover the entire ostium of the LAA, and/or the cavity may not have the depth or orientation to accept the frame of the device. These expectations appear to have been realized in the clinical trial described in the article mentioned above, wherein the device could not be deployed in approximately 1 in 10 cases. Similar assumptions underlie the symmetric barbed structure described in the above patent to Frazier et al., in that device employed to implement the purse-string method described in that patent may obtain inadequate purchase if the ostium of the LAA is irregularly shaped. In addition, the discoordinated atrial wall motion associated with atrial fibrillation may cause the foregoing percutaneously delivered devices to become dislodged during atrial fibrillation, thus posing a significant risk of thrombus release from within the previously isolated LAA.

Similar drawbacks may exist for previously-known methods and apparatus that use a pericardial approach. For example, devices that employ a loop applied to the base of the LAA on the pericardial surface may, due to normal atrial wall motion, abrade the pericardial surface, thus leading to potentially fatal cardiac pericarditis or pericardial tamponade. The clamping load applied by such previously-known loops to the base of the LAA also may significantly reduce blood flow and interfere with electrical conduction through the atrial wall in the isolated region, which in turn may result in tissue necrosis and a weakened region of the atrial wall. In addition, such previously-known apparatus and methods present a high risk of thrombus release in the event that the loop fractures or becomes dislodged.

An alternative approach, described with respect to FIGS. 14-17 and 23 of U.S. Pat. No. 6,689,150 to Van Tassel et al. involves using a pair of expandable disks to clamp and collapse the LAA tissue. As described in that patent, the expandable disks are coupled by a spring having a contracted, unstressed position. A distal end of a catheter is inserted percutaneously through the ostium and interior of the LAA and advanced until it pierces the apex of the LAA; the first expandable disk is then deployed so that it contacts the pericardial surface. An expandable filter disk is then deployed in the left atrium so that the filter disk engages the endocardial surface surrounding the ostium of the LAA. The patent describes that when the device is released from the delivery catheter, the force of the spring causes the two expandable disks to approximate, thereby causing the LAA tissue disposed between the two disks to compress and collapse the LAA. The patent further mentions, but does not provide any detail with respect to, an embodiment in which the spring could be replaced by an elastic tether, and could include teeth and a pawl to form a ratchet mechanism to pull the expandable disks towards one another.

Like various other embodiments of previously-known LAA occlusion systems noted above, the foregoing device described in the Van Tassel patent contemplates that the LAA is reasonably symmetric and has a well-defined depth and anatomy. For example, because the spring or elastic tether employed in that device will tend to cause the filter disk to become centered in the ostium of the LAA, that filter disk may not entirely occlude the ostium, making it possible for thrombus disposed in the LAA to be ejected into the left atrium. Further, is it possible that if the LAA does not have sufficient depth, the tissue will not fully clamp the tissue when the spring or elastic tether is fully contracted, thus creating the risk that the filter disk will shift during normal cardiac wall motion and periodically permit direct communication between the interior of the LAA and left atrium.

In view of the above-noted drawbacks, and others, of previously-known apparatus and methods for excluding the LAA, there remains a need for a robust percutaneous or minimally invasive method and apparatus for isolating or excluding the LAA that reduces the risk of thrombus formation in, and release from, the left atrial appendage. More particularly, there is a need for a device for excluding the LAA that enables the LAA tissue to be collapsed and permanently clamped in a preferred condition by applying a predetermined amount of load to the LAA tissue.

Percutaneous systems are known for treating atrial septal defects that permit two expandable members to be positively fastened to one another across a thickness of tissue, as described, for example, in Hausdorf, et. al., “Transcatheter closure of secundum atrial septal defects with the atrial septal defect occlusion system (ASDOS): initial experience in children”, Heart 1996:75:83-88 (1996). The device described in that article consists of left and right atrial umbrellas that include mating male and female threads. The left and right atrial umbrellas are delivered to opposing sides of the atrial septum using a guide wire loop that passes up the femoral vein, through the septal defect and exits through the femoral artery. In this manner, the two umbrellas are advanced from opposite ends of the guide wire loop until they meet at the septal defect, where a conus on the guide wire is used to retain the left atrial umbrella in position while a screwdriver catheter is engaged with the right atrial umbrella to couple the mating threads.

Although the guide wire loop described in the foregoing article provides a practical mode of approximating and coupling the left and right atrial umbrellas used in the ASDOS system, it will be immediately evident that no such a system can be employed in clamping the LAA because there is no convenient transluminal path that permits the LAA to be approached via the pericardial surface.

U.S. Pat. No. 4,007,743 to Blake describes a similar septal defect closure device including left and right atrial umbrellas and that permits deployment with single-sided access, but the device described in that patent lacks the capability to adjust the distance between the umbrellas to adapt to varied thicknesses. Accordingly, there is a need for a robust percutaneous or minimally invasive method and apparatus for isolating or excluding the LAA by deploying opposing clamping members to the endocardium of the left atrium and the pericardial surfaces of the LAA via a single percutaneous transluminal pathway.

### III. SUMMARY OF THE INVENTION

The present invention provides apparatus and methods for excluding and reducing the volume of the left atrial appendage (“LAA”) to reduce the risk of thrombus formation and release from the LAA during atrial or after atrial fibrillation. The apparatus and methods are contemplated for use on all types of LAA anatomies, including those where the ostium to the LAA is irregular and/or where the LAA cavity has a shallow depth and/or extends at an acute angle relative away from the surrounding atrial wall. In accordance with one aspect of the invention, the LAA cavity is substantially reduced in volume or eliminated by collapsing or compressing the tissue that makes up the LAA against the atrial wall and then permanently retaining the tissue in that collapsed or compressed state with a predetermined load.

In some embodiments, the atrial wall tissue forming the LAA cavity is engaged at the endocardial surface adjacent to the ostium and at the pericardial surface of the LAA, and the tissue captured therebetween is then compressed to eliminate the internal volume of the LAA cavity. In a preferred embodiment, when so compressed, the interior surface of the LAA cavity is disposed adjacent to and occludes the ostium of the LAA, so that the LAA tissue moves in synchrony with the surrounding atrial wall tissue. In addition, the elements that contact the LAA at the pericardial surface and the endocardial surface adjacent to the ostium of the LAA preferably are delivered and linked to one another using a single transluminal percutaneous or transpericardial pathway.

The apparatus of the present invention may be designed for percutaneous, minimally invasive, or surgical approaches. In some embodiments designed for percutaneous treatment, the apparatus first and second tissue capture elements and a catheter configured for transluminal insertion into the left atrium to deliver the first and second tissue capture elements. The first tissue capture element is configured for deployment in contact with the pericardium, while the second tissue engaging surface is configured to engage the endocardial surface adjacent to the ostium of the LAA. In some embodiments, the first and second tissue capture elements are arranged to be deployed before being translated towards one another, thereby compressing the LAA tissue therebetween. In other embodiments, the first tissue capture element is deployed, the apparatus is placed in traction to collapse and compress the LAA tissue, and then the second tissue capture element is deployed to retain the LAA in a compressed state. In some embodiments of the invention, the first and second tissue surfaces may be interlocked with one another to retain the LAA in a compressed state, and then decoupled from the catheter. In other embodiments, the first and second tissue capture elements are preformed so as to be linked together.

In alternative embodiments, designed for minimally invasive use, the first and second tissue capture elements may be delivered through the pericardial surface intraoperatively, or via trocar. The first tissue capture element is configured to be deployed in engagement with the endocardium adjacent to the ostium of the LAA, while the second tissue capture element is configured to engage the pericardial surface of the LAA. The first and second tissue capture elements may be preformed to be linked together, or positively engaged with one another after deployment, and then decoupled from the elongated shaft.

Methods are reducing or eliminating the volume of a LAA also are provided.

### IV. BRIEF DESCRIPTION OF THE DRAWINGS

FIG. 1 is schematic illustration of a human heart.

FIGS. 2A and 2B are cross-sectional and plan views, respectively, of the LAA.

FIG. 3 is a perspective view of a first embodiment of a device for reducing and excluding the LAA.

FIGS. 4A-4C depict the device of FIG. 3 mounted on a delivery catheter and illustrate steps of manipulating the delivery catheter to deploy the device of FIG. 3.

FIGS. 5A-5C illustrate steps of deploying the device of FIG. 3 with a transluminally positioned delivery catheter to reduce and occlude the LAA.

FIG. 6 is a perspective view of an intraoperative version of a device for reducing and excluding the LAA.

FIGS. 7A and 7B depict the device of FIG. 6 mounted on a delivery apparatus and illustrate steps of manipulating delivery apparatus to deploy the device of FIG. 6.

FIG. 8 shows the device of FIG. 6 deployed intraoperatively to reduce and occlude the LAA.

FIG. 9 is a perspective view of an alternative embodiment of a device for reducing and excluding the LAA.

FIGS. 10A-10C illustrate steps of manipulating delivery apparatus to deploy the device of FIG. 8.

FIGS. 11A and 11B are, respectively, side and plan views of a further alternative embodiment of the device of the present invention wherein the first and second tissue capture elements are foamed from a wire mesh braid so as to be linked together with a predetermined spacing.

FIG. 12 illustrates the device of FIG. 11 disposed in a delivery catheter.

FIG. 13 depicts the device of FIG. 11 deployed to reduce and occlude the LAA.

### V. DETAILED DESCRIPTION OF THE INVENTION

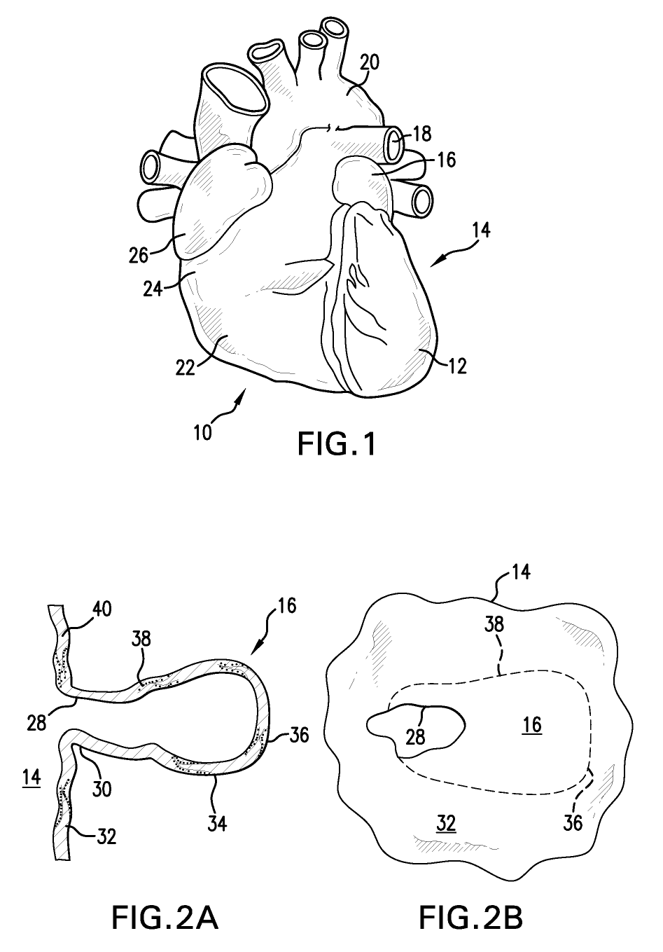

Referring to FIG. 1, heart 10 is illustrated to show certain portions including left ventricle 12, left atrium 14, left atrial appendage (LAA) 16, pulmonary artery 18, the aorta 20, the right ventricle 22, the right atrium 24, and the right atrial appendage 26. As is understood in the art, the left atrium 14 is located above the left ventricle 12 and the two are separated by the mitral valve (not illustrated). The LAA 16 is normally in fluid and electrical communication with the left atrium 14 such that blood flows in and out of the LAA, and electrical impulses conduct to and from the LAA 16 as the heart 10 beats.

FIGS. 2A and 2B are a schematic cross section of LAA 16 and a plan view of the ostium to the LAA. The chamber of the left atrium 14 and the interior of LAA 16 are shown in communication via ostium 28. The LAA is further defined as having base portion 30 where it attaches to pericardial surface 32 of the left atrium 14, and body portion 34 distal to the point of attachment of LAA 16 with the left atrium, including apex 36. Walls 38 of LAA 16 are vascularized heart tissue substantially similar to the walls 40 of the left atrium. As shown in FIG. 2B, ostium 28 may have an irregular circumference, and body portion 34 of the LAA may extend from the left atrium at a shallow angle, making it difficult to implant a circular occlusive member within the LAA.

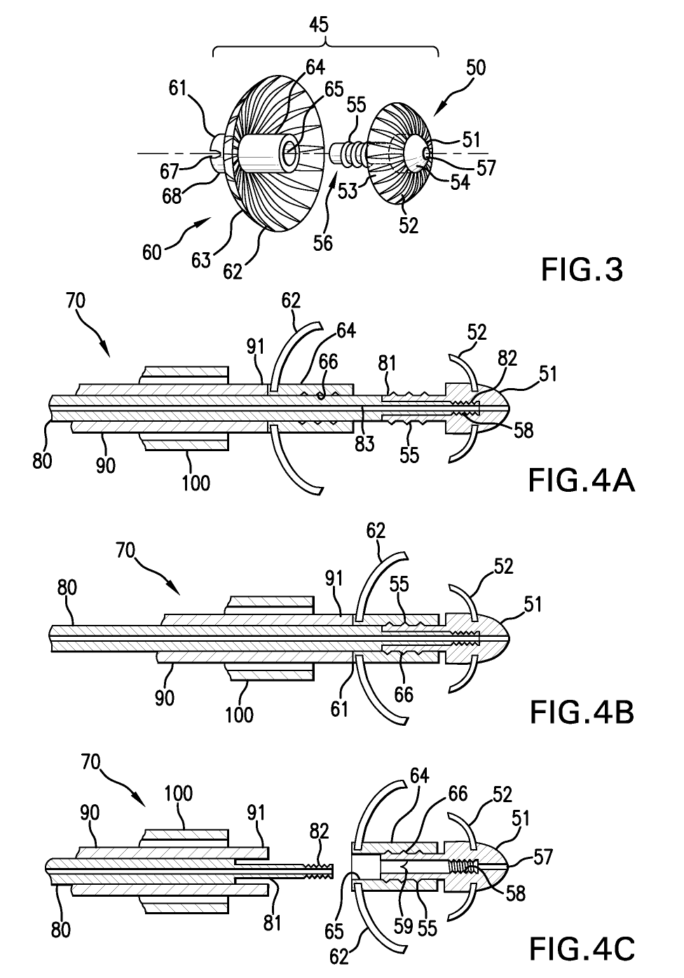

Referring now to FIG. 3, a first embodiment of device 45 for reducing and occluding a LAA, such as LAA 16, is described. Device 45 includes a pair of tissue capture elements—pericardial disk 50 and endocardial disk 60—that interengage so as to compress and collapse the LAA, and to retain the LAA in the collapsed position with a predetermined load.

Pericardial disk 50 comprises base 51 having plurality of resilient struts 52, and biocompatible cover 53 fastened to the resilient struts 52. Base 51 preferably includes an atraumatic bullet-shaped distal end 54, plurality of ribs 55 disposed on proximal portion 56, and lumen 57. Resilient struts 52, which may be formed from a biocompatible steel, biocompatible polymer or superelastic alloy, such as nickel-titanium, preferably are affixed to base 51 near distal end 54, and are configured to self-expand from a delivery state in which the struts as disposed substantially adjacent to base 51 to a deployed configuration, in which the plurality of struts extend substantially perpendicularly from base 51. As shown in FIG. 3, struts 52 may be arcuate when deployed with a proximally-directed concavity, thereby enhancing contact with the pericardial surface. Biocompatible cover 53 may comprise a flexible but strong biocompatible material, such as polyethylene, nylon or a metal alloy mesh and may be fluid impermeable or fluid permeable to serve as a filter.

Endocardial disk 60 comprises base 61, plurality of resilient struts 62, and biocompatible cover 63 fastened to the resilient struts 62. Base 61 preferably includes distal portion 64 having lumen 65 having plurality of circumferential recesses 66 that mate with ribs 55 on base 51 of pericardial disk 50, and slots 67. Resilient struts 62, which may be formed from a biocompatible steel, biocompatible polymer or superelastic alloy, such as nickel-titanium, preferably are affixed to base 61 near proximal end 68, and are configured to self-expand from a delivery state in which the struts as disposed substantially adjacent to base 61 to a deployed configuration, in which the plurality of struts extend substantially perpendicularly from base 61. As shown in FIG. 3, struts 62 may be arcuate when deployed with a distally-directed concavity, thereby enhancing contact with the endocardial surface. Biocompatible cover 63 may comprise a flexible but strong biocompatible material, such as polyethylene, nylon or a metal alloy mesh, and may be fluid impermeable or may include pores to encourage tissue ingrowth.

As will be apparent to one of ordinary skill in the art, the struts employed on the endocardial and pericardial disks may be of different or equal sizes. In addition, a self-expanding wire mesh, as used for example, in previously-known embolic filters or septal defect closure systems, may be substituted for the struts and biocompatible cover arrangement described herein without departing from the scope of the present invention. As will further be apparent to one of ordinary skill, the use of interlocking ribs 55 and recesses 66 is intended to be exemplary, and other interlocking structures, such as mating threads, bumps, mechanical fastening means, such as biocompatible adhesives, may be used to interlock the bases of the endocardial and pericardial disks.

As further depicted in FIG. 4C, disks 50 and 60 are dimensioned so that base 51 of pericardial disk 50 telescopes within base 61 of endocardial disk 60, and ribs 55 of base 51 engage circumferential recesses 66 of base 61. In this manner, endocardial disk 60 may be permanently coupled to pericardial disk 50 to apply a selected load to tissue captured therebetween, as described further below. Preferably, struts 52 are affixed adjacent to distal end 54, while struts 62 are affixed to base 61 near proximal end 68. Proximal portion 56 of base 51 and distal portion 64 of base 61 preferably are sized so that bases 51 and 61 interengage over a range of distances for reducing or occluding the LAA suitable for treating a large portion of the patient population.

Referring to FIGS. 4A to 4C, delivery catheter 70 configured for delivering device 45 via a single percutaneous transluminal pathway is described. Delivery catheter 70 includes inner member 80, tube 90 and sheath 100.

Inner member 80 includes stepped distal region 81 having threads 82 that mate with threads 58 disposed in lumen 57 of base 51 of pericardial disk 50. Inner member 80 preferably comprises a polymer typically used in catheter construction, and distal region 81 may be formed, for example, by pressing or bonding a threaded metal alloy sleeve onto a stepped end of the member. Inner member 80 additionally includes guide wire lumen 83, which permits inner member 80 to be advanced along a standard guide wire. As will of course be understood, inner member has a length, e.g., 30 cm, suitable for percutaneously accessing the right atrium via the femoral vein, and includes a suitable proximal end (not shown) for manipulation by a clinician.

Tube 90 is formed of materials conventionally used in catheter construction and includes lumen 91 dimensioned to slide freely over the exterior of inner member 80. Tube 90 includes plurality of projections 91 that interengage with slots 67 in proximal end 68 of base 61. Tube 90 preferably has a length comparable to that of inner member 80, and includes a suitable proximal end (not shown) for manipulation by a clinician.

Sheath 100 also is formed of materials conventionally used in catheter construction and includes lumen 101 dimensioned to slide freely over the exterior of tube 90. When advanced distally over tube 90 and inner member 80, sheath 100 causes plurality of struts 62 and biocompatible cover 63 on endocardial disk 60, and plurality of struts 52 and biocompatible cover 53 on pericardial disk 50, to transition to a contracted delivery state. When sheath 100 is retracted proximally, as described below, the struts of disks 50 and 60 assume deployed states. Sheath 100 preferably has a length sufficient to cover tube 90 and inner member 80 when advanced distally, and includes a suitable proximal end (not shown) for manipulation by a clinician.

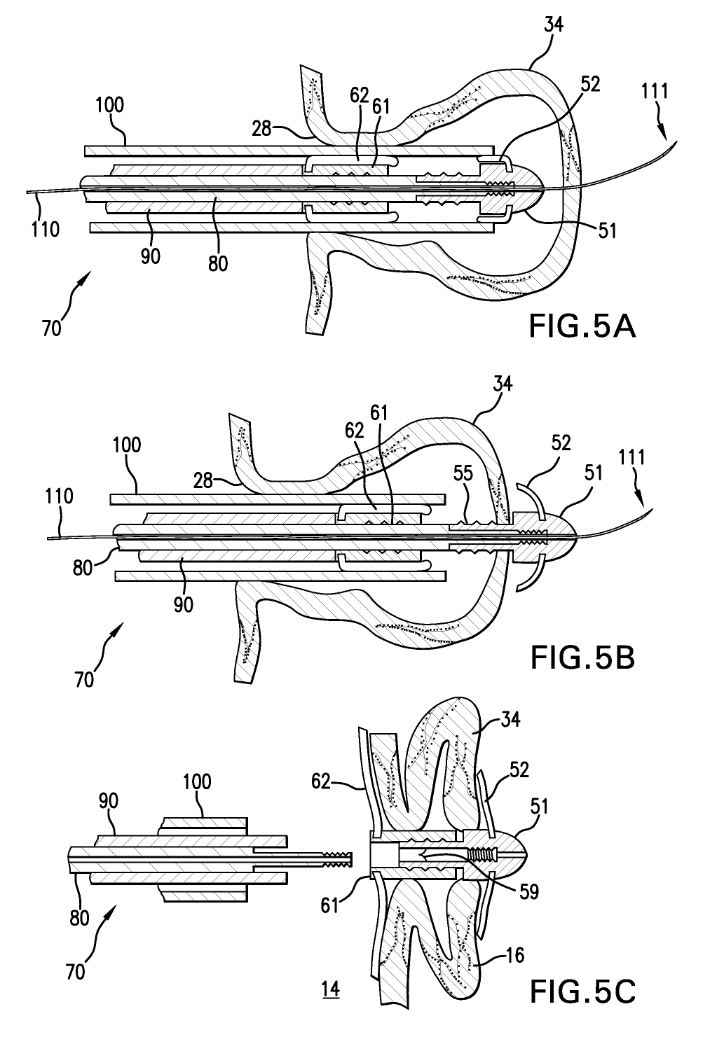

In FIGS. 4A through 4C, operation of delivery catheter 70 to deploy device 45 is described; steps of using delivery catheter 70 to deploy device 45 to reduce and occlude a LAA are describe with respect to FIGS. 5A-5C.

In FIG. 4A, pericardial disk 50 is shown mounted on distal region 81 of inner member 80, with threads 58 of lumen 57 in proximal portion 56 of base 51 engaged with threads 82 of distal region 81. Tube 90 is shown with its distal end abutted against proximal end 68 of endocardial disk 60, with both displaced proximally from pericardial disk 50. Mating threads 58 and 82 secure the pericardial disk to the delivery catheter so that, after the pericardial disk has been inserted through an aperture in the wall of the LAA and deployed, endocardial disk 60 may be advanced distally to drive distal portion 64 of base 61 over proximal portion 56 of base 51 until one or more ribs 55 engage recesses 66, thereby locking disks 50 and 60 together, as shown in FIG. 4B.

Once disks 50 and 60 are positively engaged, tube 90 is held stationary with projections 91 engaged with slots 67. Inner member 80 then is rotated to unscrew threads 82 from mating threads 58 in base 51 of pericardial disk 50. As will of course be apparent, keeping projections 91 engaged with slots 67 in the proximal end of the base 61 ensures that the entire device 45 does not rotate when the clinician attempts to unscrew inner member from base 51. Once the pericardial disk is decoupled from the inner member, delivery catheter 70 may be removed. As further illustrated in FIG. 4C, lumen 57 of base 51 may include membrane 59 that forms a one-way valve that prevents blood from passing through lumen 57 into the pericardial space when inner member 80 is decoupled from base 51 of the pericardial disk.

With respect to FIGS. 5A to 5C, a method of employing device 45 and delivery catheter 70 to reduce and occlude a LAA is now described. In a first step, guide wire 110 having sharpened tip 111 (preferably within a flexible atraumatic sheath, not shown) is advanced via a cutdown through the femoral vein or by standard percutaneous access techniques, and into the right atrium under fluoroscopic guidance. Using the standard transeptal technique with a Mullins sheath or similar, and a Brockenbrough needle (or any other type of needle such as Ross etc, or even using standard RF-transeptal device catheter) or also using tip 111 of a sharp guide wire may then be exposed to permit the guide wire to pierce the atrial septum. Tip 111 of guide wire 110 is then directed so that it passes through the ostium 28 of LAA 16, and pierces wall 38 of the LAA. The wire is then advanced within the pericardial sac and rapped around the heart for further stability. The wire may be exchanged for an extra-support type of wire and then the delivery catheter is advanced over the wire within the pericardial sac. Device 45, preloaded onto delivery catheter 70, then is advanced along guide wire 110 until it is disposed within LAA, as depicted in FIG. 5A.

With respect to FIG. 5B, delivery catheter 70 and device 45 are advanced into the pericardial sac over guide wire 110 until bullet-shaped distal end 54 of base 51 passes through the aperture made by guide wire 110 in wall 38 and struts 53 and biocompatible cover 53 (not shown) deploy beyond the pericardial surface of the LAA within the pericardial sac. Next, delivery catheter 70 is retracted proximally until pericardial disk 50 contacts the pericardial surface. Delivery catheter 70 then is retracted proximally until endocardial disk 60 is disposed within the left atrium, and sheath 100 is retracted proximally so that struts 62 transition from the contracted delivery state to the expanded state.

Next, inner member 80 is held stationary while tube 90 is advanced distally, thereby cause proximal portion 56 of base 51 of pericardial disk 50 to telescope within lumen 65 of base 61 of endocardial disk 60. As the two components are coupled together, for example, under fluoroscopic imaging, the clinician may experience an increasing decree of friction as ribs 55 of base 51 engage recesses 66 in base 61, during which the LAA is collapsed upon itself. After the clinician has driven disks 50 and 60 together so that the intervening tissue of the LAA is fully collapsed, tube 90 is held stationary with its projections 91 engaged with slots 67 in base 61 while inner member 80 is rotated to disconnect device 45 from the delivery catheter. As shown in FIG. 5C, once inner member 80 disengages from device 45, delivery catheter 70 may be withdrawn from the left atrium.

Advantageously, because disks 50 and 60 are rigidly and permanently coupled together, there is expected to be little risk that endocardial disk 60 could become dislodged or shift due to cardiac wall motion. In addition, the system described above enables the clinician to deliver and deploy device 45 via a single percutaneous transluminal pathway.

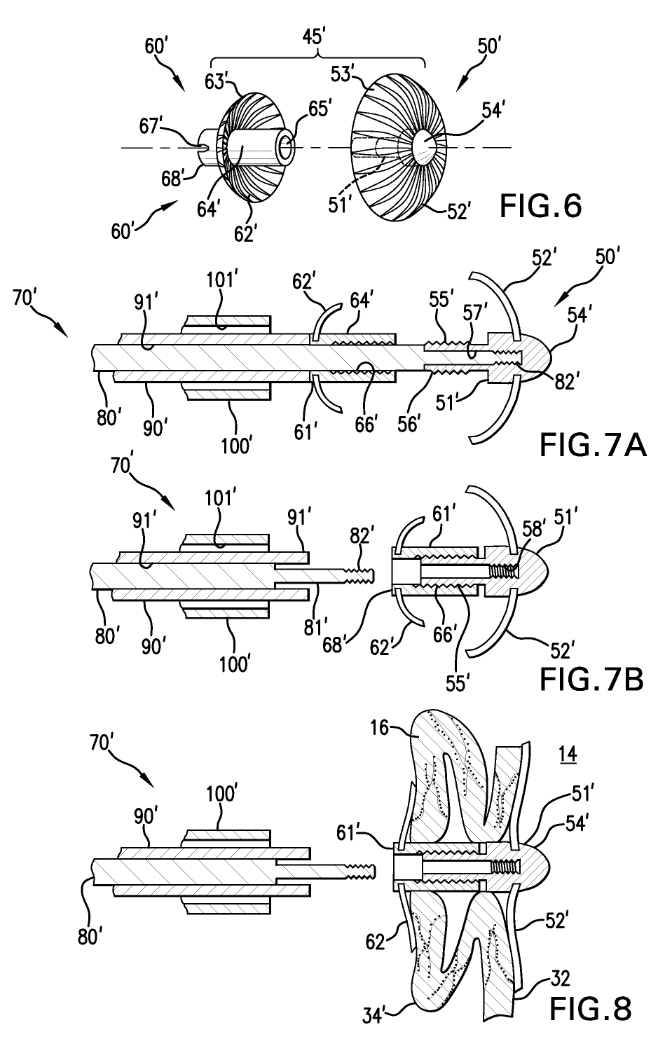

As will be apparent to one of ordinary skill, device 45 and delivery catheter 70 of FIGS. 3 and 4 may be readily adapted for intraoperative or minimally invasive surgical use. Referring now to FIGS. 6 through 8, an alternative embodiment of the device and delivery apparatus of the present invention suitable for use intraoperatively or with minimally invasive techniques is described. In the embodiment of FIGS. 6-8, components similar to those described with respect to the embodiments of FIGS. 3-5 are designated with like-prime numbers. Thus for example, endocardial disk 60 is denoted as 60′.

Referring now to FIG. 6, device 45′ for reducing and occluding a LAA using intraoperative techniques is described. Device 45′ includes endocardial disk 50′ and pericardial disk 60′ that interengage so as to compress and collapse the LAA, and to retain the LAA in the collapsed position with a predetermined load. Device 45′ is similar to device 45 of FIG. 3, except that device 45′ is applied from the pericardial surface inward, rather than from the left atrium outward. Accordingly, the distal portion of device 45′ comprises the endocardial disk, and preferably includes longer struts that contact a larger area, while proximal portion of device 45′ comprises the pericardial disk, and may include smaller struts. As will be apparent to one of ordinary skill in the art, the struts employed on the endocardial and pericardial disks may be of different or equal sizes. In addition, a self-expanding wire mesh, as used for example, in previously-known embolic filters or septal defect closure systems, may be substituted for the struts and biocompatible cover arrangement described herein without departing from the scope of the present invention.

Endocardial disk 50′ comprises base 51′ having plurality of resilient struts 52′, and biocompatible cover 53′ fastened to the resilient struts 52′. Base 51′ preferably includes an atraumatic bullet-shaped distal end 54′, plurality of ribs 55′ disposed on proximal portion 56′, and lumen 57′. Resilient struts 52′, which may be formed from a biocompatible steel, biocompatible polymer or superelastic alloy, such as nickel-titanium, preferably are affixed to base 51′ near distal end 54′, and are configured to self-expand from a delivery state in which the struts as disposed substantially adjacent to base 51′ to a deployed configuration, in which the plurality of struts extend substantially perpendicularly from base 51′. As shown in FIG. 6, struts 52′ may be arcuate when deployed with a proximally-directed concavity, thereby enhancing contact with the endocardial surface. Biocompatible cover 53′ may comprise a flexible but strong biocompatible material, such as polyethylene, nylon or a metal alloy mesh and may be fluid impermeable or fluid permeable to serve as a filter.

Pericardial disk 60′ comprises base 61′, plurality of resilient struts 62′, and biocompatible cover 63′ fastened to the resilient struts 62′. Base 61 preferably includes distal portion 64′ having lumen 65′ having plurality of circumferential recesses 66′ that mate with ribs 55′ on base 51′ of endocardial disk 50′, and slots 67′. Resilient struts 62′, which may be formed from a biocompatible steel, biocompatible polymer or superelastic alloy, such as nickel-titanium, preferably are affixed to base 61′ near proximal end 68′, and are configured to self-expand from a delivery state in which the struts as disposed substantially adjacent to base 61′ to a deployed configuration, in which the plurality of struts extend substantially perpendicularly from base 61′. As shown in FIG. 6, struts 62′ may be arcuate when deployed with a distally-directed concavity, thereby enhancing contact with the pericardial surface. Biocompatible cover 63′ may comprise a flexible but strong biocompatible material, such as polyethylene, nylon or a metal alloy mesh, and may be fluid impermeable or may include pores to encourage tissue ingrowth.

As further depicted in FIGS. 7A and 7C, disks 50′ and 60′ are dimensioned so that base 51′ of endocardial disk 50′ telescopes within base 61′ of pericardial disk 60′, and ribs 55′ of base 51′ engage circumferential recesses 66′ of base 61′. In this manner, pericardial disk 60′ may be permanently coupled to endocardial disk 50′ to apply a selected load to tissue captured therebetween, as described further below. Preferably, struts 52′ are affixed adjacent to distal end 54′, while struts 62′ are affixed to base 61′ near proximal end 68′. Proximal portion 56′ of base 51′ and distal portion 64′ of base 61′ preferably are sized so that bases 51′ and 61′ interengage over a range of distances for reducing or occluding the LAA suitable for treating a large portion of the patient population.

Referring to also FIGS. 7A to 7C, delivery apparatus 70′ configured for delivering device 45′ from an exterior approach to the heart, either through a suitably positioned trocar or during a surgical procedure, is described. Delivery apparatus 70′ includes inner member 80′, tube 90′ and sheath 100′.

Inner member 80′ includes stepped distal region 81′ having threads 82′ that mate with threads 58′ disposed in lumen 57′ of base 51′ of endocardial disk 50′. Inner member 80′ preferably comprises a metal alloy or polymer typically used in medical device construction, and distal region 81′ may be formed, for example, by pressing or bonding a threaded metal alloy sleeve onto a stepped end of the member. As will of course be understood, inner member has a length suitable for accessing the LAA and left atrium via a pericardial approach, and includes a suitable proximal end (not shown) for manipulation by a clinician.

Tube 90′ is formed of materials conventionally used in medical device construction and includes lumen 91′ dimensioned to slide freely over the exterior of inner member 80′. Tube 90′ includes plurality of projections 91′ that interengage with slots 67′ in proximal end 68′ of base 61′. Tube 90′ preferably has a length comparable to that of inner member 80′, and includes a suitable proximal end (not shown) for manipulation by a clinician.

Sheath 100′ also is formed of materials conventionally used in medical device construction and includes lumen 101′ dimensioned to slide freely over the exterior of tube 90′. When advanced distally over tube 90′ and inner member 80′, sheath 100′ causes plurality of struts 62′ and biocompatible cover 63′ on pericardial disk 60′, and plurality of struts 52′ and biocompatible cover 53′ on endocardial disk 50′, to transition to a contracted delivery state. When sheath 100′ is retracted proximally, as described below, the struts of disks 50′ and 60′ assume deployed states. Sheath 100′ preferably has a length sufficient to cover tube 90′ and inner member 80′ when advanced distally, and includes a suitable proximal end (not shown) for manipulation by a clinician.

As shown in FIG. 7A, endocardial disk 50′ is shown mounted on distal region 81′ of inner member 80′, with threads 58′ of lumen 57′ in proximal portion 56′ of base 51′ engaged with threads 82′ of distal region 81′. Tube 90′ is shown with its distal end abutted against proximal end 68′ of pericardial disk 60′, with both displaced proximally from endocardial disk 50′. Mating threads 58′ and 82′ secure the endocardial disk to the delivery apparatus, so that after endocardial disk 50 has been inserted through an aperture in the wall of the LAA and deployed, pericardial disk 60 may be advanced distally to drive distal portion 64′ of base 61′ over proximal portion 56′ of base 51′ until one or more ribs 55′ engage recesses 66′, thereby locking disks 50′ and 60′ together, as shown in FIG. 7B.

Once disks 50′ and 60′ are positively engaged, tube 90′ is held stationary with projections 91′ engaged with slots 67′. Inner member 80′ then is rotated to unscrew threads 82′ from mating threads 58′ in base 51′ of endocardial disk 50′. As will of course be apparent, keeping projections 91′ engaged with slots 67′ in the proximal end of the base 61′ ensures that the entire device 45′ does not rotate when the clinician attempts to unscrew inner member from base 51′. Once the endocardial disk is decoupled from the inner member, delivery apparatus 70′ may be removed.

With respect to FIG. 8, a final step of intraoperative operation of delivery apparatus 70′ to deploy device 45′ to reduce and occlude a LAA is described. In a first step, LAA 16 is exposed, either by thoracotomy or by placing one or more trocars and visualization devices adjacent to the heart. A conventional surgical device may then be used to pierce the wall of the LAA. Delivery apparatus 70′ and device 45′ then are manipulated so that bullet-shaped distal end 54′ of base 51′ passes through the aperture in the wall of the LAA and struts 53′ and biocompatible cover 53′ (not shown) deploy within the left atrium to contact tissue surrounding the ostium of the LAA. Next, delivery apparatus 70′ is retracted proximally until endocardial disk 50′ contacts the endocardial surface and occludes the ostium to the LAA. Delivery apparatus 70′ then is retracted proximally until pericardial disk 60′ is disposed adjacent to the pericardial surface of the left atrium, and sheath 100′ is retracted proximally so that struts 62′ transition from the contracted delivery state to the expanded state.

Next, inner member 80′ is held stationary while tube 90′ is advanced distally, thereby cause proximal portion 56′ of base 51′ of endocardial disk 50′ to telescope within lumen 65′ of base 61′ of pericardial disk 60′. As the two components are coupled together, the clinician may experience an increasing decree of friction as ribs 55′ of base 51′ engage recesses 66′ in base 61′, during which the LAA is collapsed upon itself. After the clinician has driven disks 50′ and 60′ together so that the intervening tissue of the LAA is fully collapsed, tube 90′ is held stationary with its projections 91′ engaged with slots 67′ in base 61′ while inner member 80′ is rotated to disconnect device 45′ from the delivery apparatus. As shown in FIG. 8, once inner member 80′ disengages from device 45′, delivery apparatus 70′ may be removed. Preferably, when device 45′ is fully deployed, the LAA is collapsed against the pericardial surface of the left atrium, and moves in synchrony with the left atrial wall.

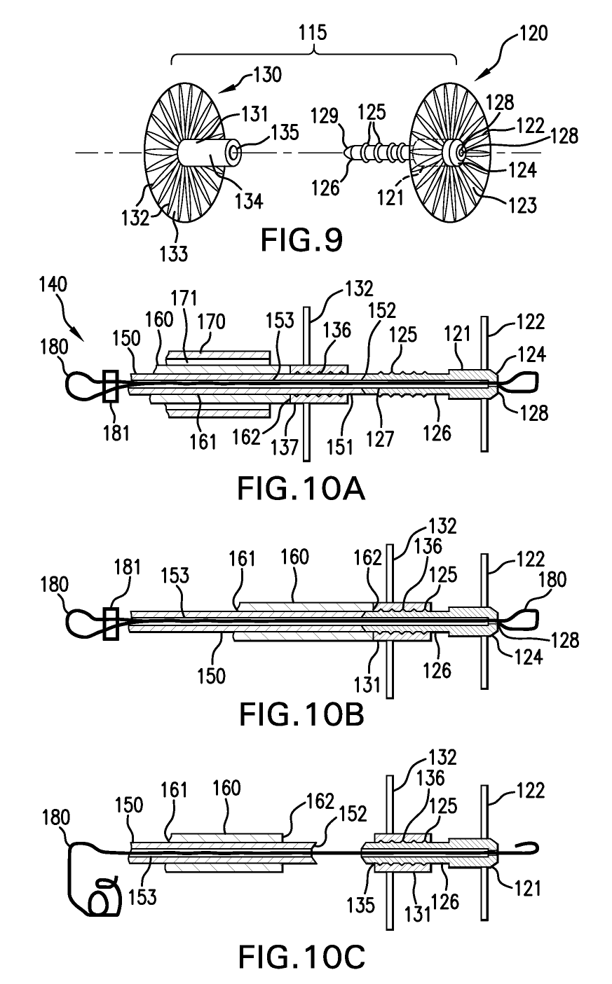

Referring now to FIGS. 9 and 10, a second embodiment of the present invention is described comprising two self-expanding disks that be deployed via a transluminal approach from the left atrium or an intraoperative or minimally invasive approach from the pericardial surface. As in the preceding embodiments, one disk is deployed in contact with the pericardial surface, the other is deployed to span of occlude the ostium of the LAA, and the two disks are drawn together and coupled to retain the LAA tissue in a collapsed, occluded configuration.

Referring now to FIGS. 9 and 10, device 115 for reducing and occluding a LAA, such as LAA 16, is described. Device 115 includes pericardial disk 120 and endocardial disk 130 that interengage so as to compress and collapse the LAA, and to retain the LAA in the collapsed position with a predetermined load.

Pericardial disk 120 comprises base 121 having plurality of resilient struts 122, and biocompatible cover 123 fastened to the resilient struts 122. Base 121 preferably includes atraumatic distal end 124, plurality of ribs 125 disposed on proximal portion 126, lumen 127, pair of apertures 128 and beveled proximal end 129. Resilient struts 122, which may be formed from a biocompatible steel, biocompatible polymer or superelastic alloy, such as nickel-titanium, preferably are affixed to base 121 near distal end 124, and are configured to self-expand from a delivery state in which the struts as disposed substantially adjacent to base 121 to a deployed configuration, in which the plurality of struts extend substantially perpendicularly from base 121. As shown in FIG. 9, struts 122 may be straight or, as in prior embodiments, arcuate when deployed. Biocompatible cover 123 may comprise a flexible but strong biocompatible material, such as polyethylene, nylon or a metal alloy mesh and may be fluid impermeable or fluid permeable to serve as a filter. Apertures 128 communicate with lumen 127, and may be spaced equidistant across the endface of distal end 124, or offset, in which case one of the pair may also serve as a guide wire lumen.

Endocardial disk 130 comprises base 131, plurality of resilient struts 132, and biocompatible cover 133 fastened to the resilient struts 132. Base 131 preferably includes distal portion 134 having lumen 135 having plurality of circumferential recesses 136 that mate with ribs 125 on base 121 of pericardial disk 120. Resilient struts 132, which may be formed from a biocompatible steel, biocompatible polymer or superelastic alloy, such as nickel-titanium, preferably are affixed to base 131 near proximal end 137, and are configured to self-expand from a delivery state in which the struts as disposed substantially adjacent to base 131 to a deployed configuration, in which the plurality of struts extend substantially perpendicularly from base 131. As shown in FIG. 9, struts 132 may extend perpendicularly from base 131, although other configurations, such as an arcuate shape illustrated with respect to preceding embodiments may be employed. Biocompatible cover 133 may comprise a flexible but strong biocompatible material, such as polyethylene, nylon or a metal alloy mesh, and may be fluid impermeable or may include pores to encourage tissue ingrowth.

As will be apparent to one of ordinary skill in the art, the struts employed on the endocardial and pericardial disks may be of different or equal sizes. In addition, a self-expanding wire mesh, as used for example, in previously-known embolic filters or septal defect closure systems, may be substituted for the struts and biocompatible cover arrangement described herein without departing from the scope of the present invention. As will further be apparent to one of ordinary skill, the use of interlocking ribs and recesses is intended to be exemplary, and other interlocking structures, such as mating threads, bumps, mechanical fastening means, such as biocompatible adhesives, may be used to interlock the bases of the endocardial and pericardial disks.

As further depicted in FIG. 10C, disks 120 and 130 are dimensioned so that base 121 of pericardial disk 120 telescopes within base 131 of endocardial disk 130, and ribs 125 of base 121 engage circumferential recesses 136 of base 131. In this manner, endocardial disk 130 may be permanently coupled to pericardial disk 120 to apply a selected load to tissue captured therebetween, as described for the preceding embodiments. Preferably, struts 122 are affixed adjacent to distal end 124, while struts 132 are affixed to base 131 near proximal end 137, so as to minimize the extent to which the bases protrude into the left atrium and pericardial spaces, respectively. Proximal portion 126 of base 121 and distal portion 134 of base 131 preferably are sized so that bases 121 and 131 interengage over a range of distances for reducing or occluding the LAA suitable for treating a large portion of the patient population.

Referring now also to FIGS. 10A to 10C, delivery apparatus 140 configured for delivering device 115 is described. As will be apparent from inspecting the similarities between the embodiments of FIGS. 3 and 6 above, delivery apparatus 140 may be readily configured to deliver device 115 via either a single percutaneous transluminal pathway, or an intraoperative or minimally invasive approach, is described. Delivery apparatus 140 includes inner member 150, tube 160, sheath 170, and high strength suture or wire 180.

Inner member 150 includes distal end 151 having inwardly-beveled endface 152 configured to abut against beveled endface 129 of base 121, and lumen 153. Suture or wire 180 runs in a continuous loop through lumen 153 from the proximal end of delivery apparatus, where it can be manipulated by the clinician, to base 121, where individual strands of the loop pass through apertures 128. By virtue of this arrangement, a clinician may apply a proximally-directed force to base 121 by pulling wire or suture 180 proximally. Inner member 150 preferably comprises a polymer or metal alloy typically used in medical device construction. As will of course be understood, inner member 150 has a length suitable for the desired mode or delivery, and includes a suitable proximal end (not shown) for manipulation by a clinician.

Tube 160 is formed of materials conventionally used in medical device construction and includes lumen 161 dimensioned to slide freely over the exterior of inner member 150. Tube 160 includes endface 162 that abuts against proximal end 137 of base 131. Tube 160 preferably has a length comparable to that of inner member 150, and includes a suitable proximal end (not shown) for manipulation by a clinician.

Sheath 170 also is formed of materials conventionally used in medical device construction and includes lumen 171 dimensioned to slide freely over the exterior of tube 160. When advanced distally over tube 160 and inner member 150, sheath 170 causes plurality of struts 132 and biocompatible cover 133 on endocardial disk 130, and plurality of struts 122 and biocompatible cover 123 on pericardial disk 120, to transition to a contracted delivery state. When sheath 170 is retracted proximally, as described below, the struts of disks 120 and 130 assume deployed states. Sheath 170 preferably has a length sufficient to cover tube 170 and inner member 150 when advanced distally, and includes a suitable proximal end (not shown) for manipulation by a clinician.

With respect to FIGS. 10A through 10C, operation of delivery catheter 140 to deploy device 115 is now described. In FIG. 10A, pericardial disk 120 is shown engaged to the distal end 152 of inner member 150, with suture or wire 180 extending through lumens 153, 127 and apertures 128. Tube 160 is shown with its distal end abutted against proximal end 137 of endocardial disk 130, with both displaced proximally from pericardial disk 120. Suture or wire 180 secures the pericardial disk to inner member 150, using for example, clip or clamp 181 applied to the proximal portion of the loop formed in suture or wire 180, so as to keep the suture or wire taut in lumens 153 and 127.

As shown in FIG. 10B, once the pericardial disk has been inserted through an aperture in the wall of the LAA and deployed, sheath 170 is retracted proximally to permit the struts of endocardial disk to deploy. The clinician then holds suture or wire 180 taut while the endocardial disk is advanced distally by pushing tube 160 in the distal direction. This action drives base 131 over proximal portion 126 of base 121 until one or more ribs 125 engage recesses 136, thereby locking disks 120 and 130 together, as shown in FIGS. 10B and 10C. This action also causes the distance between disks 120 and 130 to decrease, collapsing and compressing the LAA tissue, while the endocardial disk occludes the ostium of the LAA.

Once disks 120 and 130 are positively engaged, clip or clamp 181 is removed, and suture or wire 180 is cut at the proximal end of the device. The clinician then pulls suture or wire 180 through apertures 128 in base 121, thereby decoupling device 115 from the delivery apparatus. Once device 115 is decoupled from the inner member, delivery apparatus 140 may be removed. As for the preceding embodiment, when device 115 is fully deployed, the LAA is collapsed against the pericardial surface of the left atrium, and moves in synchrony with the left atrial wall.

Advantageously, because disks 120 and 130 are rigidly and permanently coupled together, there is expected to be little risk that endocardial disk 130 could become dislodged or shift due to cardiac wall motion. In addition, the system described above enables the clinician to deliver and deploy device 115 via a single percutaneous transluminal pathway, or intraoperative or minimally invasive pathway.

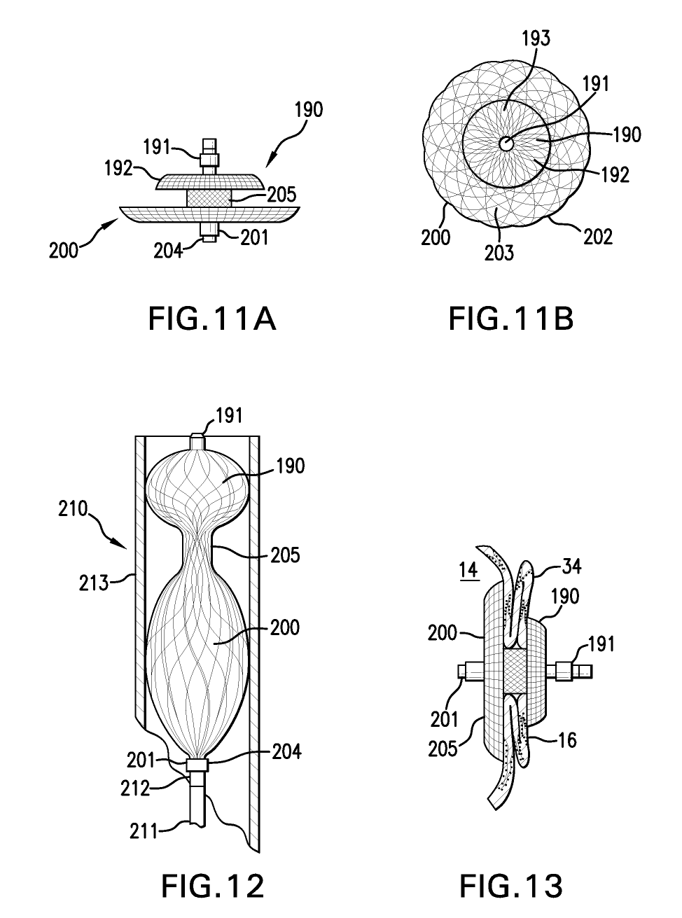

Referring now to FIGS. 11 to 13, a further alternative embodiment of the apparatus of the present invention is described. Device 185 for reducing and occluding a LAA, such as LAA 16, includes a pair of tissue capture elements—pericardial disk 190 and endocardial disk 200—that compress and collapse the LAA, and retain the LAA in the collapsed position with a predetermined load.

Pericardial disk 190 comprises base 191 coupled to plurality of resilient wires 192 woven into a braid that self-expands to a predetermined, preformed shape as disclosed in U.S. Pat. No. 5,725,552 to Kotula et al., the entirety of which is incorporated herein by reference. Pericardial disk 190 may include optional biocompatible membrane 193 fastened to the resilient wires 192. Base 191 provides a termination that retains resilient wires 192 properly braided and oriented, and prevents the distal end of the braid from fraying. Resilient wires 192 may be formed from a biocompatible steel, biocompatible polymer or superelastic alloy, such as nickel-titanium, and are configured to self-expand from a contracted delivery state to a deployed configuration, as depicted in FIGS. 11A and 11B. As shown in FIG. 11A, pericardial disk 190 may be preformed to assume a proximally-directed concave shape when deployed, thereby enhancing contact with the pericardial surface. Biocompatible cover 193 may comprise a flexible but strong biocompatible material, such as polyethylene, nylon or a metal alloy mesh and may be fluid impermeable or fluid permeable to serve as a filter.

Endocardial disk 200 comprises base 201, plurality of resilient wires 202, and optional biocompatible membrane 203 fastened to the resilient wires 202. Wires 202 are arranged in a braid that assumes a preformed shape when deployed, as discussed in the aforementioned U.S. Pat. No. 5,725,552. Base 201 preferably includes proximal portion 204 having a threaded lumen that accepts a mating threaded component of the delivery system. Resilient wires 202, which may be formed from a biocompatible steel, biocompatible polymer or superelastic alloy, such as nickel-titanium, preferably are affixed to base 201 and are configured to self-expand from a contracted delivery state to a deployed configuration, as depicted in FIGS. 11A and 11B. As shown in FIG. 11A, wires 202 may be preformed to assume a distally-directed concave shape when deployed, thereby enhancing contact with the endocardial surface. Biocompatible cover 203 may comprise a flexible but strong biocompatible material, such as polyethylene, nylon or a metal alloy mesh, and may be fluid impermeable or may include pores to encourage tissue ingrowth.

Wires 192 and 202 that form pericardial disk 190 and endocardial disk 200, respectively, are continuous strands of wire, and preferably in addition form link 205 that serves to separate disks 190 and 200 by a predetermined distance in the deployed state. Endocardial and pericardial disks may be of different or equal sizes. In a preferred embodiment, pericardial disk has an expanded diameter in a range of 10 mm to 15 mm, while endocardial disk has a diameter of about 24 to 32 mm. Preferably, the endocardial disk will overlap the endocardial tissue surrounding the ostium to the LAA by about 3 mm, and thus may be provided in a range of sizes from 24 to 32 mm in 2 mm increments. Link 205 may have a length, e.g., 3-4 mm, selected so as to clamp the collapsed tissue of the LAA with a predetermined load when implanted as described hereinbelow. In addition, base 191 may be omitted from the pericardial disk by making the device as described in U.S. Patent Publication No. 2007/0043391 A1 to Moszner et al., the entirety of which is incorporated herein by reference. As a further alternative, the device may include an internal locking mechanism, as described in U.S. Pat. No. 5,853,422 to Huebsch et al. or U.S. Patent Publication No. 2005/0273135 to Chandusko et al., the entireties of which are incorporated herein by reference.

Referring now to FIG. 12, delivery system 210 configured for delivering device 185 is described. Delivery system 210 includes pushrod 211 having threaded distal end 212, and sheath 213, and may be configured to deliver device 185 via a single percutaneous transluminal pathway, intraoperatively, or via a minimally invasive pericardial approach.

Pushrod 211 includes threaded distal end 212 that mates with threads disposed in base portion 204 of base 201. Pushrod 211 preferably comprises a torquable metal alloy wire or polymer, as typically used in catheter construction, has a length appropriate for the selected delivery method, and a proximal end (not shown) for manipulation by a clinician. Sheath 213 also is formed of materials conventionally used in medical device construction, and includes lumen 214 dimensioned to permit device 185 to be slidably disposed in lumen 214 in a contracted delivery state. When advanced distally over device 185 and pushrod 211, sheath 213 causes endocardial disk 200 and pericardial disk 190 to transition to a contracted delivery state. When sheath 213 is retracted proximally, as described below, disks 190 and 200 assume deployed states. Sheath 213 preferably has a length sufficient to cover pushrod 211 when advanced distally, and includes a suitable proximal end (not shown) for manipulation by a clinician.

Operation of delivery system 210 to deploy device 185 is now described. Preferably, the patients' LAA is first imaged to determine the approximate size of the LAA tissue mass, and the approximate size of the ostium of the LAA. Device 185 having appropriately selected dimensions then is selected. Preferably, device 185 is prepackaged in a sterile container disposed in sheath 213 and coupled at base 201 to pushrod 211.

A guide wire having a sharpened tip, such as described above with respect to the methods depicted in FIGS. 5A to 5C, is advanced via a cutdown through the femoral vein and into the right atrium under fluoroscopic guidance. The guide wire pierces the atrial septum, and is then directed so that it passes through the ostium of the LAA, and pierces the wall of the LAA. Next, device 185, preloaded in delivery system 210, is advanced alongside the guide wire until it is disposed within LAA. The distal end of the delivery system then is advanced through the aperture made by the guide wire and into the pericardial space, where sheath 213 is retracted proximally to deploy pericardial disk beyond the pericardial surface of the LAA.

Next, the clinician applies a proximally directed force to delivery system 210 to first cause pericardial disk 190 to contact the pericardial surface. Delivery system 210 then is pulled further in the proximal direct to cause the LAA to compress and collapse upon itself. Sheath 213 then is retracted proximally until endocardial disk 200 deploys in the left atrium and occludes the ostium of the LAA, as depicted in FIG. 13. As illustrated in FIG. 13, when deployed in the manner described above, device 185 retains the tissue of the LAA in a compressed state with a predetermined load selected based on the length of link 205.

After endocardial disk 200 deploys in the left atrium, device 185 applies a sufficiently high load to the compressed LAA that threaded region 212 of pushrod 211 may be unscrewed from portion 204 of base 201, thereby decoupling device 185 from pushrod 211. Delivery system 210 then is withdrawn from the left atrium, and if needed, a atrial septal defect device may be deployed to plug the trans-atrial access path created by guide wire. As for the preceding embodiments, when device 185 is fully deployed, the LAA preferably is collapsed against the pericardial surface of the left atrium, and moves in synchrony with the left atrial wall.

Advantageously, because disks 190 and 200 are permanently coupled together, there is expected to be little risk that endocardial disk 200 could become dislodged or shift due to cardiac wall motion. In addition, the system described above enables the clinician to deliver and deploy device 185 via a single percutaneous transluminal pathway. As will be apparent to one of ordinary skill, device 185 and delivery catheter 210 of FIGS. 11-13 may be readily adapted for intraoperative or minimally invasive surgical use. In this case, base 191 may include a threaded lumen to engage threaded region 212 of pushrod 211, and the orientation of device may be reversed when device 185 is loaded into sheath, i.e., so that endocardial disk is delivered into the left atrium first, followed by collapsing the LAA and deploying pericardial disk 200 in the pericardial space to complete the implantation.

While various illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed:

1. Apparatus for reducing volume of a patient's left atrial appendage comprising:
an implantable device comprising first and second tissue capture elements;
the first tissue capture element configured to transition between a contracted delivery configuration and an expanded configuration, the first tissue capture element configured to engage a pericardial surface of the patient's left atrial appendage in the expanded configuration;
the second tissue capture element configured to transition between a contracted delivery configuration and an expanded configuration, the second tissue capture element configured to engage endocardial tissue surrounding an ostium of the patient's left atrial appendage in the expanded configuration; and
a delivery system configured to deploy the implantable device so that first and second tissue capture elements cause the patient's left atrial appendage to collapse therebetween, the first and second tissue capture elements rigidly coupled together in the deployed state to apply a predetermined load that retains the patient's left atrial appendage in a compressed, reduced volume state.

2. The apparatus of claim 1 wherein the first and second tissue capture elements comprise disks formed of a preformed wire braid, and the first and second tissue capture elements are rigidly coupled together by a link formed from continuous strands of the preformed wire braid.

3. The apparatus of claim 2 wherein the link has a length of between 2 to 6 mm.

4. The apparatus of claim 1 wherein the first tissue capture element comprises a first base, the second tissue capture element comprises a second base, and the first and second tissue capture elements become rigidly coupled together when the first base interlocks with the second base.

5. The apparatus of claim 1 wherein the delivery system is a catheter configured for transluminal placement in the left atrial appendage, the first tissue capture element is disposed on a distal end of the catheter and the second tissue capture element is disposed proximal of the first tissue capture element.

6. The apparatus of claim 1 wherein the delivery system comprises an elongated shaft configured for minimally invasive surgical use, the second tissue capture element is disposed on a distal end of the elongated shaft and the first tissue capture element is disposed on the elongated shaft proximal of the second tissue capture element.

7. The apparatus of claim 1 wherein at least one of the first and second tissue capture elements comprises an expandable disk.

8. The apparatus of claim 1 wherein at least one of the first and second tissue capture elements comprises a plurality of struts.

9. The apparatus of claim 1 wherein at least one of the first and second tissue capture elements comprises a self-expanding mesh.

10. The apparatus of claim 1 wherein at least one of the first and second tissue capture elements comprises a superelastic material.

11. The apparatus of claim 1 wherein at least one of the first and second tissue capture elements comprises a biocompatible, implantable membrane.

12. The apparatus of claim 1 wherein the delivery system comprises an inner member and a tube configured for relative movement therebetween, the first tissue capture element releasably coupled to the inner member.

13. The apparatus of claim 1 wherein the first and second tissue capture elements are of unequal size in the expanded configurations.

14. The apparatus of claim 1 wherein the first tissue capture element assumes a concave, convex or flat shape in the expanded configuration.

15. The apparatus of claim 4 wherein the first base defines an elongated portion having a plurality of ribs, and the second base defines a lumen having a plurality or recesses, at least one of the plurality of ribs engaging at least one of the plurality of recesses when the first based interlocks with second base.

16. The apparatus of claim 1 wherein one of the first and second tissue capture elements includes a threaded portion that mates with a threaded portion on the delivery system.

17. The apparatus of claim 1 wherein the delivery system comprises a suture or wire that releasably couples the first or second tissue capture element to the delivery system.

18. Apparatus for reducing volume of a patient's left atrial appendage comprising:
a first tissue capture element configured to transition between a contracted delivery configuration and an expanded configuration, the first tissue capture element configured to engage a pericardial surface of the patient's left atrial appendage in the expanded configuration;
a second tissue capture element configured to transition between a contracted delivery configuration and an expanded configuration, the second tissue capture element configured to engage endocardial tissue surrounding an ostium of the patient's left atrial appendage in the expanded configuration;
wherein the first and second tissue capture elements are configured to be approximated to collapse the patient's left atrial appendage therebetween and to retain the patient's left atrial appendage in a compressed, reduced volume state by application of a predetermined load.

19. The apparatus of claim 18 wherein at least one of the first and second tissue capture elements comprises a self-expanding disk.

20. The apparatus of claim 18 wherein at least one of the first and second tissue capture elements comprises a plurality of struts.

21. The apparatus of claim 18 wherein at least one of the first and second tissue capture elements comprises a self-expanding preformed wire braid.

22. The apparatus of claim 18 wherein at least one of the first and second tissue capture elements comprises a superelastic material.

23. The apparatus of claim 18 wherein at least one of the first and second tissue capture elements comprises a biocompatible, implantable membrane.

24. The apparatus of claim 18 wherein the first and second tissue capture elements are of unequal size in the expanded configurations.

25. A method of reducing volume of a patient's left atrial appendage comprising:
providing a first tissue capture element;
providing a second tissue capture element;
expanding the first tissue capture surface to engage a pericardial surface of the patient's left atrial appendage;
collapsing the patient's left atrial appendage; and
expanding the second tissue capture element to engage endocardial tissue surrounding an ostium of the patient's left atrial appendage,
whereby the patient's left atrial appendage is retained in a compressed, reduced volume state.

26. The method of claim 25 wherein collapsing the patient's left atrial appendage comprises applying traction to the first tissue capture element.

27. The method of claim 25 wherein the first and second tissue capture elements are delivered transluminally.

28. The method of claim 25 further comprising decoupling the first and second tissue capture elements from a delivery system.

29. The method of claim 28 wherein one of the first and second tissue capture elements includes a threaded portion that mates with a threaded portion of the delivery system, and decoupling the first and second tissue capture elements from the delivery system comprises unscrewing the mating threaded portions.

30. The method of claim 25 wherein expanding the second tissue capture element is performed before collapsing the patient's left atrial appendage.

31. The method of claim 30 wherein the first tissue capture element comprises a first base and the second tissue capture element comprises a second base, the method further comprising coupling the first base to the second base.

32. The method of claim 31 wherein the first base defines an elongated portion having a plurality of ribs, and the second base defines a lumen having a plurality of recesses, wherein coupling the first base to the second base comprises engaging at least one of the plurality of ribs with at least one of the plurality of recesses.

33. The method of claim 28 wherein the delivery system comprises a suture or wire that releasably couples the first tissue capture element or second tissue capture element to the delivery system, and decoupling the first and second tissue capture elements from the delivery system comprises cutting the suture or wire.

34. A method of reducing volume of a patient's left atrial appendage comprising:
providing a first tissue capture element;
providing a second tissue capture element;
expanding the first tissue capture surface to engage an endocardial surface of the patient's left atrium surrounding an ostium of the patient's left atrial appendage;
collapsing the patient's left atrial appendage; and
expanding the second tissue capture element to engage pericardial tissue,
whereby the patient's left atrial appendage is retained in a compressed, reduced volume state.

35. The method of claim 34 wherein collapsing the patient's left atrial appendage comprises applying traction to the first tissue capture element.

36. The method of claim 34 wherein the first and second tissue capture elements are delivered from the pericardial surface.

37. The method of claim 36 further comprising decoupling the first and second tissue capture elements from a delivery system.

38. The method of claim 37 wherein one of the first and second tissue capture elements includes a threaded portion that mates with a threaded portion of the delivery system, and decoupling the first and second tissue capture elements from the delivery system comprises unscrewing the mating threaded portions.

39. The method of claim 34 wherein expanding the second tissue capture element is performed before collapsing the patient's left atrial appendage.

40. The method of claim 39 wherein the first tissue capture element comprises a first base and the second tissue capture element comprises a second base, the method further comprising coupling the first base to the second base.

41. The method of claim 40 wherein the first base defines an elongated portion having a plurality of ribs, and the second base defines a lumen having a plurality of recesses, wherein coupling the first base to the second base comprises engaging at least one of the plurality of ribs with at least one of the plurality of recesses.

42. The method of claim 37 wherein the delivery system comprises a suture or wire that releasably couples the first tissue capture element or second tissue capture element to the delivery system, and decoupling the first and second tissue capture elements from the delivery system comprises cutting the suture or wire.
